(19) United States
(12) Patent Application Publication
(10) Pub. No.: US 2024/0245546 A1
(43) Pub. Date: Jul. 25, 2024

(54) IN-LINE ORAL DEVICE FOR SLEEP APNEA

(71) Applicant: Ana C. Krieger, New York, NY (US)

(72) Inventor: Ana C. Krieger, New York, NY (US)

(21) Appl. No.: 18/420,584

(22) Filed: Jan. 23, 2024

Related U.S. Application Data
(60) Provisional application No. 63/481,016, filed on Jan. 23, 2023.

(51) Int. Cl. A61F 5/56

(52) U.S. Cl. CPC A61F 5/566

(57) ABSTRACT

An oral device for use by a patient during sleep can include a first guard and a second guard. The first guard can be configured for insertion into an oral cavity of the patient and can include a first body configured to engage a first set of a dentition and a first recess. The second guard can be configured for insertion into the oral cavity of the patient and can include a second body configured to engage a second set of the dentition and a first projection. The first projection can extend from the second body and can align with a first set of occlusal surfaces of the second set of the dentition upon insertion of the second guard.

### CLAIM OF PRIORITY

This patent application claims the benefit of priority, under 35 U.S.C. Section 119(e), to Ana Krieger, U.S. Patent Application Ser. No. 63/481,016 entitled “IN-LINE ORAL DEVICE FOR SLEEP APNEA,” filed on Jan. 23, 2023, which is hereby incorporated by reference herein in its entirety.

### BACKGROUND

Sleep apnea is a common disorder in the United States and internationally. One of the most prevalent types of sleep apnea is obstructive sleep apnea, where those affected can have problems breathing during sleep, which can cause other health problems and potentially death. Obstructive sleep apnea is often caused by incorrectly functioning soft tissue and upper airway muscles during sleep and is often treated using continuous positive airway pressure (CPAP) machines or mouth guards or splints.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 illustrates a perspective view of an oral cavity of a patient and an oral device.

FIG. 2 illustrates a side view of a dentition of a patient and an oral device.

FIG. 3 illustrates a side view of a dentition of a patient and an oral device.

FIG. 4A illustrates a side view of an oral device.

FIG. 4B illustrates a side view of an oral device.

FIG. 4C illustrates a side view of an oral device.

FIG. 5 illustrates a side view of an oral device.

FIG. 6 illustrates a side view of an oral device.

FIG. 7 illustrates a bottom perspective view of a portion an oral device.

FIG. 8 illustrates a bottom view of a portion an oral device.

FIG. 9 illustrates a top view of a portion an oral device.

### DETAILED DESCRIPTION

Mouth guards or splints are often used to treat sleep apnea and especially obstructive sleep apnea to help improve breathing and to help reduce snoring during sleep. However, these guards can be relatively difficult to use to obtain a natural position of the jaw or mandible while maintaining an unobstructed airway for the user. Also, positioning or alignment features used with guards are often located laterally outward of the dentition which can cause discomfort and irritation.

The devices of this application help to address these issues by using a dual plate cover of the upper and lower teeth that can be used to open the mouth and advance the mandible. The plates or guards can include features forming an in-line connection to create an opening between the upper and lower teeth, helping to provide a natural position of the dentitions with respect to each other while maintaining an unobstructed airway for the user. Because the features are in-line with the dentition, discomfort from use can be minimized as the appliance would occupy a gap in space created by the opening of the mouth.

Also, mandibular advancement has been determined to be effective as a first line option for treating milder cases of obstructive sleep apnea. However, many devices try to minimize a gap and limit mandibular anterior movement or advancement to address apnea or snoring. The devices of this application help to promote minor mandibular advancement and to form a relatively small gap between dentitions to help address apnea or snoring.

The above discussion is intended to provide an overview of subject matter of the present patent application. It is not intended to provide an exclusive or exhaustive explanation of the invention. The description below is included to provide further information about the present patent application.

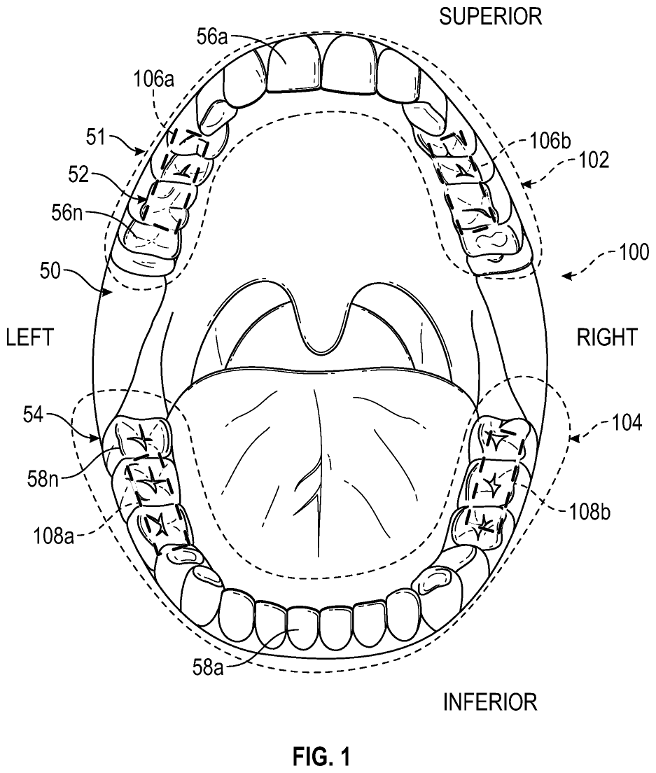

FIG. 1 illustrates a perspective view of an oral cavity 50 of a patient and an oral device 100 to help treat sleep apnea. The oral cavity 50 can include a superior half (or set) 52 of a dentition 51 and an inferior half (or set) 54 of the dentition 51. The superior half 52 can include teeth 56a-56n and the inferior half 54 can include teeth 58a-58n. FIG. 1 also shows orientation indicators Superior, Inferior, Left, and Right (from an observer's perspective).

The device 100 can include a superior guard 102 (or upper guard) and an inferior guard 104 (or lower guard). The superior guard 102 can be configured for insertion into the oral cavity 50 of the patient and be configured to engage the superior half 52 such as to engage and at least partially surround the teeth 56. Similarly, the inferior guard 104 can be configured for insertion into the oral cavity 50 of the patient and be configured to engage the inferior half 54 such as to engage and at least partially surround the teeth 58.

The superior guard 102 can include a first connection feature 106a and a second connection feature 106b. Similarly, the inferior guard 104 can include a third connection feature 108a and a fourth connection feature 108b. The features can be configured to engage each other when the patient closes his or her jaw. For example, the first connection feature 106a can engage the third connection feature 108a and the second connection feature 106b can engage the fourth connection feature 108b. Such engagement between the connection features can position the superior guard 102 and the inferior guard 104 with respect to each other to place the dentitions and the mandible in a natural and desired position to help promote a normal and natural airway and to thereby help limit disruptive sleep apnea.

The connection features 106 and 108 can each be a recess or projection that is in-line with its dentition, and that is in not lateral to the dentition, but lateral as to the mid-line of the dentition. For example, the first connection feature 106a can be in-line with a left set of occlusal surfaces of the superior half 52 of the dentition 51, the second connection feature 106b can be in-line with a right set of occlusal surfaces of the superior half 52 of the dentition 51, the third connection feature 108a can be in-line with a left set of occlusal surfaces of the inferior half 54 of the dentition 51 and the fourth connection feature 108b can be in-line with a right set of occlusal surfaces of the inferior half 54 of the dentition 51. That is to say, the features 106 and 108 are in-line with their respective teeth of the dentition 51 and are not located laterally outward thereof.

Other oral devices include features that are positioned laterally outward of the dentition, which can cause soft tissue irritation. Because the connection features 106 and 108 are in-line with the dentitions, the oral device 100 can help to reduce soft tissue irritation during use of the oral device 100. That is, the connection features 106 and 108 will minimally (or will not) engage with soft tissues, such as gingiva or buccal mucosa, helping to reduce irritation from use of the oral device 100. Similar oral devices are discussed in further detail below.

The superior guard 102 and the inferior guard 104 can also help to provide cushion and support for the teeth and lower mandible, while helping to keep the mouth open and the mandible advanced to the desired degree in order to treat snoring, upper airway resistance syndrome, sleep hypopnea, sleep apnea, and perhaps grinding, clenching, or the like.

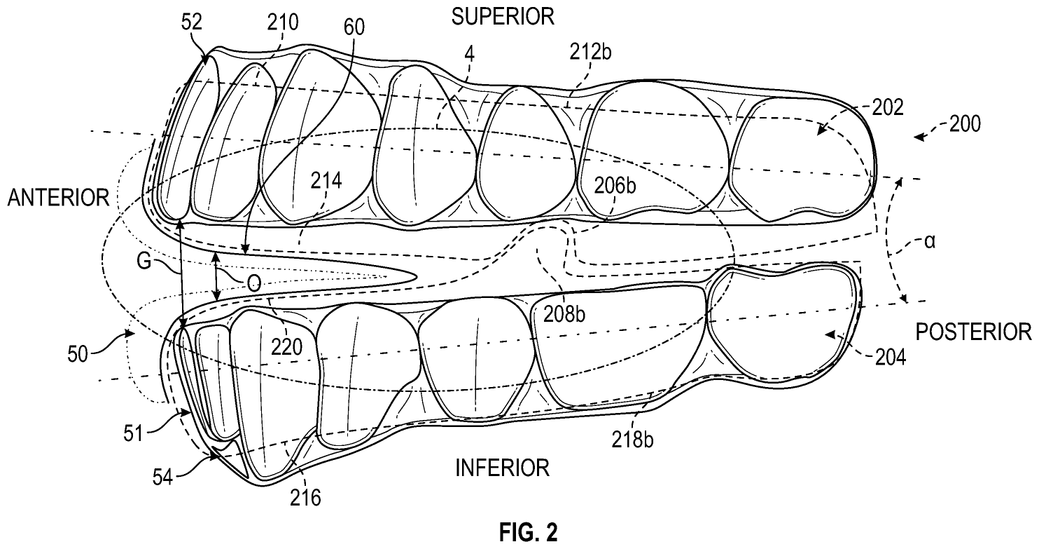

FIG. 2 illustrates a side view of a dentition of a patient and an oral device 200. FIG. 2 also shows figure indicator 4 and orientation indicators Superior, Inferior, Anterior, and Posterior. The oral device 200 can be similar to the oral device 100 discussed above. The 200 can include a projection and a recess that are in-line with the patients' dentition 51 and are configured to mate to align the jaw and dentition 51 of the patient. Any oral device discussed above or below can include these or other features discussed below.

The oral device 200 can include a superior guard 202 and an inferior guard 204. The superior guard 202 can include a superior body (or first body) 210 defining lateral extensions 212a and 212b. The superior guard 202 can also define a contact surface 214. The inferior guard 204 can include an inferior body (or second body) 216 defining lateral extensions 218a and 218b. The inferior guard 204 can also define a contact surface 220.

The superior body 210 of the superior guard 202 can define a recess 206b, which can be an in-line connection feature, meaning the recess 206b can be in-line with the teeth of the dentition 51, and not, for example, laterally outward thereof. The recess 206b can extend into the superior body 210 from the contact surface 214 and can terminate prior to reaching the superior half 52. The inferior body 216 of the contact surface 214 can define a projection 208b, which can be an in-line connection feature, meaning the projection 208b is in-line with the teeth of the dentition 51, and not, for example, laterally outward thereof. The projection 208b can extend from the contact surface 220 and can be configured (e.g., sized and shaped) for insertion into the recess 206b.

In operation or use of some examples, the patient can insert the superior guard 202 into the oral cavity 50 to engage the superior half 52 and can insert the inferior guard 204 into the oral cavity to engage the inferior half 54, as shown in FIG. 2. In a proper orientation, the projection 208b can extend superiorly from the inferior half 54 toward the superior half 52 of the dentition 51. The user can then move their mandible and the inferior half 54 to position the projection 208b into the recess 206b to position the inferior guard 104 with respect to the superior guard 102 and to position the superior half 52 of the dentition 51 with respect to the inferior half 54 of the dentition 51. Optionally, when the projection 206b is engaged with the projection 208b, the contact surface 214 can engage the contact surface 220 to limit movement of the superior half 52 of the dentition 51 with respect to the inferior half 54 of the dentition 51. The engagement between the projections 206 can also provide cushion or support between the superior and inferior teeth to help attenuate dental stress from grinding and clenching, in addition to increasing the oral cavity size which will in turn reduce the severity of snoring and sleep apnea.

The recess 206b and the projection 208b can be positioned on their respective guards such as to create a gap G between the superior half 52 and the inferior half 54 of the dentition 51 to form a natural airway to help reduce disruptive sleep apnea. For example, the gap G can be between superior incisors of the teeth 56 and inferior incisors of the teeth 58. Optionally, when the 206b is engaged with the projection 208b, the contact surface 214 can engage the contact surface 220 to help define the gap G and an opening O in a mouth 60.

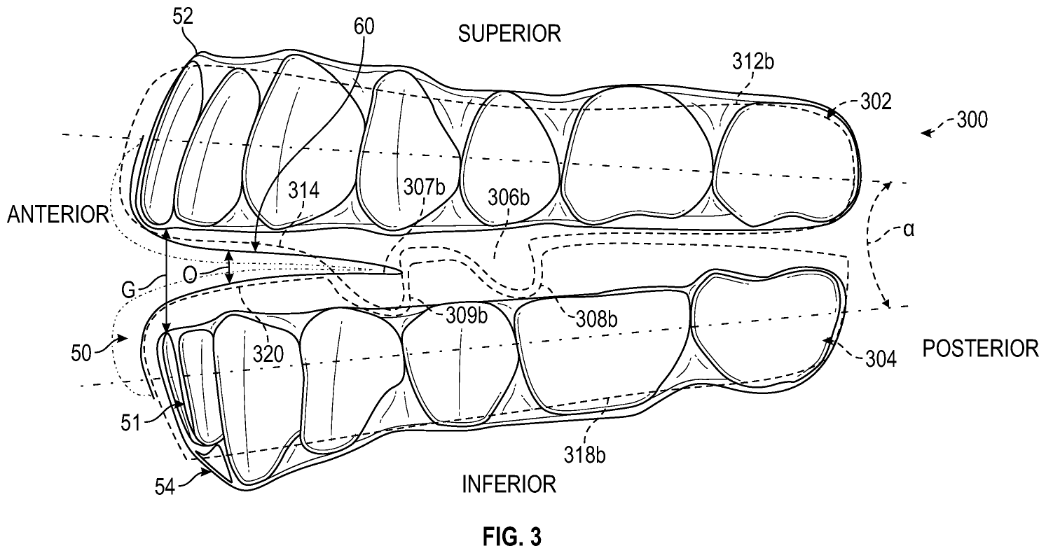

The recess 206b and the projection 208b can also be configured such that their engagement can form a natural angle of the jaw, created by the opening O of the mouth 60 (shown in FIGS. 2 and 3), which can be an angle α between the superior half 52 and the inferior half 54 of the dentition 51, which can help improve patient comfort while using the oral device 200 during sleep. Optionally, the superior guard 202 or the inferior guard 204 can be patient-matched or customized based on the patient's anatomy such that the recess 206b and the projection 208b engage to form the angle α that is most natural or comfortable for that patient.

FIG. 2 also shows that the projection 208b can be angled anteriorly and the recess 206b can be angled complimentary to the projection 208b. Such an angle between the projection 208b and the recess 206b can help to limit separation of the superior guard 202 from the inferior guard 204 during sleep. The angle between the connection features 206 and 208 in FIG. 2 is shown as being moderate, but can be more extreme (as shown in other examples below) to help limit separation of the superior guard 202 and the inferior guard 204 during use.

Optionally, the superior guard 202 can be connected to the inferior guard 204, such as through a living hinge or other connection mechanism or device that allows for relative movement between the superior guard 202 and the inferior guard 204.

FIG. 3 illustrates a side view of the dentition 51 of a patient and an oral device 300. FIG. 3 also shows orientation indicators Superior, Inferior, Anterior, and Posterior. The oral device 300 can be similar to the devices 100 and 200 discussed above. The guards of the device 300 can include multiple connection features on each lateral side of each guard. Any of the oral devices discussed above and below can include these or other features

As shown in FIG. 3, a superior guard 302 can include a superior body 210 including a first projection 306b and a second projection 307b. An inferior guard 304 can include a first recess 308b and a second recess 309b. In use of the superior guard 302 and the inferior guard 304, the first projection 306b can be insertable into the first recess 308b and the second projection 307b can be insertable into the second recess 309b to define the gap G and the angle α. Use of multiple connection features can help to increase ease of alignment and can help to increase a retention force to help limit separation of the guards during use.

Optionally, as shown in FIG. 3, the recess 309b can extend into a recess between teeth of the inferior half 54. The inferior guard 304 can optionally include such a recess to allow the recess to extend further inferiorly such as to obtain a desired angle α, a desired gap G, or to help increase retention between the superior guard 302 and the inferior guard 304. Optionally, the inferior guard 304 can be patient-matched (e.g., designed based on the patient's anatomy) to locate such a recess.

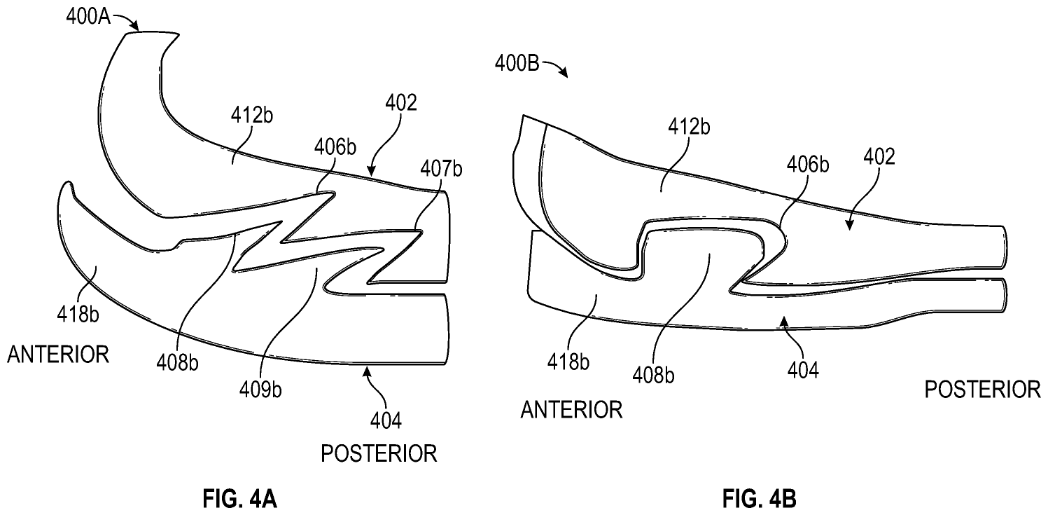

FIG. 4A illustrates a side view of an oral device 400A. The oral device 400A can be similar to the devices discussed above. The guards of the device 400A can include multiple connection features on each lateral side of each guard angled aggressively posteriorly. Any of the oral devices discussed above and below can include these or other features discussed below.

More specifically, a superior guard 402 can include recesses 406b and 407b extending into a body 412b. An inferior guard 404 can include projections 408b and 409b extending from a body 418b. The projections 408b and 409b can extend aggressively anteriorly-to-posteriorly such that the projections 408b and 409b, when engaged with the recesses 406b and 407b, respectively, can help limit separation between of the superior guard 402 from the inferior guard 404.

FIGS. 4B and 4C illustrate side views of oral device 400B and 400C. The oral devices 400B and 400C can be similar to the devices discussed above. The guards of the devices 400B and 400C can include a connection feature having an irregular shape to help limit separation. Any of the oral devices discussed above and below can include these or other features discussed below.

More specifically, a superior guard 402 can include a recess 406b extending into a body 412b. An inferior guard 404 can include a projection 408b extending from a body 418b. The projection 408b can be shaped irregularly and the recess 406b can be shaped complimentary to the projection 408b such that the projection 408b, when engaged with the recess 406b, can help limit separation between of the superior guard 402 from the inferior guard 404. For example, the projection 408b can include a portion larger than an opening of the recess, which can require deformation of the projection 408b or the recess 406b to allow insertion of the projection 408b into the recess 406b or removal of the projection 408b from the recess 406b, which can help limit unwanted separation of the superior guard 402 from the inferior guard 404 during use.

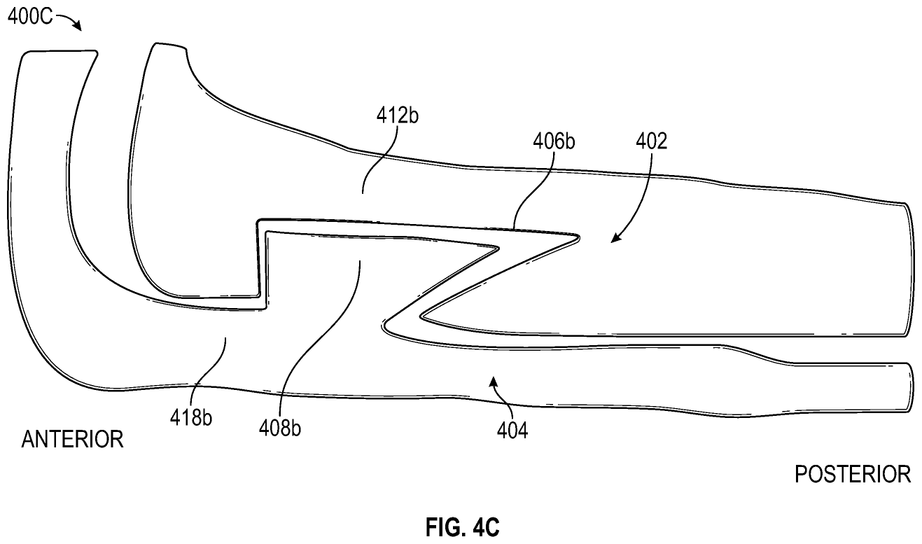

FIG. 5 illustrates a side view of an oral device 500. The oral device 500 can be similar to the devices discussed above. The guards of the device 500 can include multiple connection features on each lateral side of each guard angled aggressively posteriorly. Any of the oral devices discussed above and below can include these or other features discussed below.

The oral device 500 can include a superior guard 502 and an inferior guard 504. The superior guard 502 can include recesses 506a-506n extending into a body 512b of the guard. The inferior guard 504 can include projections 508a and 508b extending from a body 518b. The superior guard 502 can include any number of recesses 506a-506n and the inferior guard 504 can include any number of projections 508a. Optionally, by including fewer projections 508 than recesses, the inferior guard 504 can be positioned in several positions with respect to the superior guard 502, which can allow the user to find a natural jaw position during use of the device 500. The projections and recesses of the device 500 can be any of the shapes discussed above.

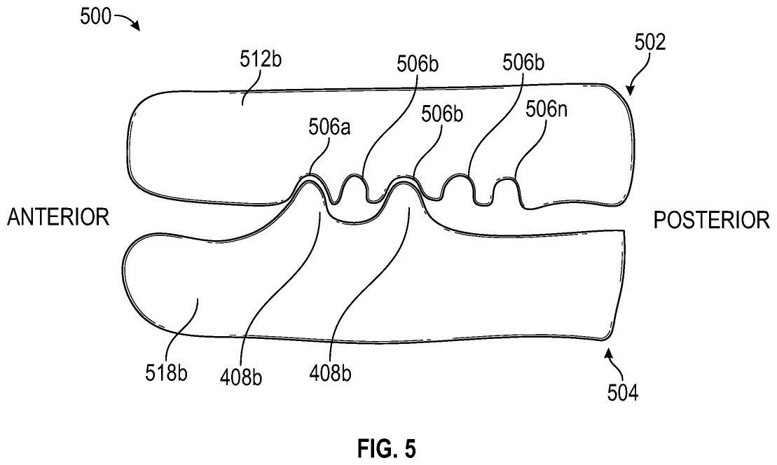

FIG. 6 illustrates a side view of an oral device 600. The oral device 600 can be similar to the devices discussed above. The guards of the device 600 can include engaging projections configured to promote anterior mandible displacement and opening or separation between the superior and inferior dentition portions. Any of the oral devices discussed above and below can include these or other features discussed below.

More specifically, the oral device 600 can include a superior guard 602 and an inferior guard 604. The superior guard 602 can include a projection 606, a recess 608, and a body 610. The inferior guard 604 can include a body 612, a projection 614, and a recess 616.

The projection 606 can extend posteriorly-to-anteriorly as it extends superiorly-to-inferiorly from the body 610 of the superior guard 602, where such extension can at least partially define the recess 608 between the body 610 and the projection 606. Similarly, projection 614 can extend anteriorly-to-posteriorly as it extends inferiorly-to-superiorly from the body 612 of the inferior guard 604, where such extension can at least partially define the recess 616 between the body 612 and the projection 614.

In use, the bodies 610 and 612 can engage each other to form a gap (e.g., gap G) between the superior dentition and the inferior dentition, which can help reduce sleep apnea, snoring, grinding, or clenching. The gap formed between the superior dentition and the inferior dentition can be filled by material of the bodies 610 and 612, which can help to significantly reduce grinding since the teeth of the superior dentition and the inferior dentition are not able to contact through the material of the bodies 610 and 612. The gap can also help reduce the effects of clenching. Though clenching may still occur, damage to the temporomandibular joints (TMJ) due to clenching can be reduced as the joints will not be able to completely close.

As shown in FIG. 6, the projection 614 can be at least partially insertable into the recess 608 and the projection 606 can be at least partially insertable into the recess 616 such that the projection 606 and the projection 614 abut or engage to limit relative movement of the superior guard 602 with respect to the inferior guard 604 when the superior guard 602 and the inferior guard 604 are inserted to engage inferior and superior portions of a dentition, respectively. In other words, the projections 606 and 614 and the recesses 608 and 616 can work together to interlock or to lock to each other. In some examples, the superior guard 602 can be inserted into the dentition and the inferior guard 604 can then be inserted such that the projections 606 and 614 and the recesses 608 and 616 interlock, helping to ensure the oral device 600 is properly positioned in the oral cavity. In other examples, the projections 606 and 614 and the recesses 608 and 616 can be interlocked prior to insertion of the superior guard 602 and the inferior guard 604 into the oral cavity, which can also help to ensure the oral device 600 is properly positioned in the oral cavity.

The projections 606 and 614 and the recesses 608 and 616 can help limit separation between of the superior guard 602 from the inferior guard 604. The projections 606 and 614 and the recesses 608 and 616 can also, together, be configured to promote anterior mandibular movement or displacement when the superior guard 602 and the inferior guard 604 are inserted to engage the superior dentition and the inferior dentition, respectively, which can help to reduce apnea, snoring, or snorting. The projections 606 and 614 and the recesses 608 and 616 can also, together, be configured to create a gap between the superior dentition and the inferior dentition to increase an overall oral cavity size. For example, given a typical anterior-posterior length of a dental arcade of 4 centimeters (cm) and a typical lateral distance of 4 cm (side-to-side space), the oral device 600 may create an increase in oral cavity size of approximately 8 cubic centimeters, which can significantly improve sleep apnea, snoring, gasping, or snorting.

The advancement of the lower jaw by an average of 2 to 6 millimeters (mm) is typically well tolerated when treating sleep apnea using mandibular advancement devices. As such, the projections 606 and 614 and the recesses 608 and 616 can be configured to advance the lower jaw (to promote anterior mandibular movement or displacement) between 2 and 6 mm. However, the projections 606 and 614 and the recesses 608 and 616 can be configured to advance the lower jaw more or less.

In some examples, the oral device 600 can be provided with superior guard 602 or inferior guard 604 of different sizes, which can allow a patient to change one or more of the guards to increase or decrease an amount that the lower jaw is advanced. For example, if a patient using the oral device 600 determines that they are still snoring or having apnea, the inferior guard 604 can be swapped with a different inferior guard (e.g., guard 604A) that can have a projection that is larger than the projection 614, which can promote increased anterior mandibular movement, to further help improve snoring or apnea. The oral device 600 can optionally come in a kit with multiple guards to change mandibular movement, such as in 1 mm increments, allowing the user to find their most effective mandible displacement. Other increments (e.g., 0.1 mm or 0.5 mm can be used).

The guards 602 and 604 can similarly be configured to change a distance between the superior dentition and the inferior dentition, which can increase or decrease an amount of space or cushion between the dentitions. For example, if a patient using the oral device 600 determines that they are still having grinding problems, the inferior guard 604 can be swapped with a different inferior guard (e.g., guard 604A) that can have a body that is thicker, which can promote increased cushion or space between dentitions, to further help improve grinding or clenching. The oral device 600 can optionally come in a kit with multiple guards to change mandibular movement, such as in 1 mm increments, allowing the user to find their most effective dentition spacing. Other increments (e.g., 0.1 mm or 0.5 mm can be used).

FIG. 6 also shows that the projections 606 and 614 and the recesses 608 and 616 can interface at an angle θ. That is the projection 606 can form an angle θ that can interface with the recess 616 at a complementary angle. The projection 614 and the recess 608 can be similarly configured. The angle θ can be between 20 degrees and 60 degrees to help interlock the superior guard 602 and the inferior guard 604 while helping to prevent separation between the superior guard 602 and the inferior guard 604. Optionally, the angle θ can be between 30 degrees and 45 degrees, such as 30 degrees, 35 degrees, 40 degrees, 45 degrees, or the like.

Though the oral device 600 shows only projection for each of the superior guard 602 and the inferior guard 604, each lateral side of the superior guard 602 and the inferior guard 604 can include a projection, such that the superior guard 602 and the inferior guard 604 can be mirrored (such as with the oral device 100).

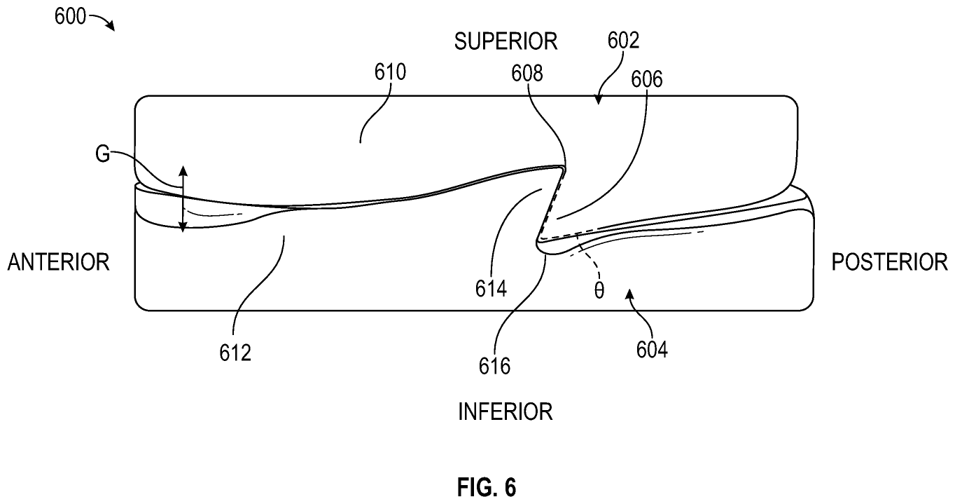

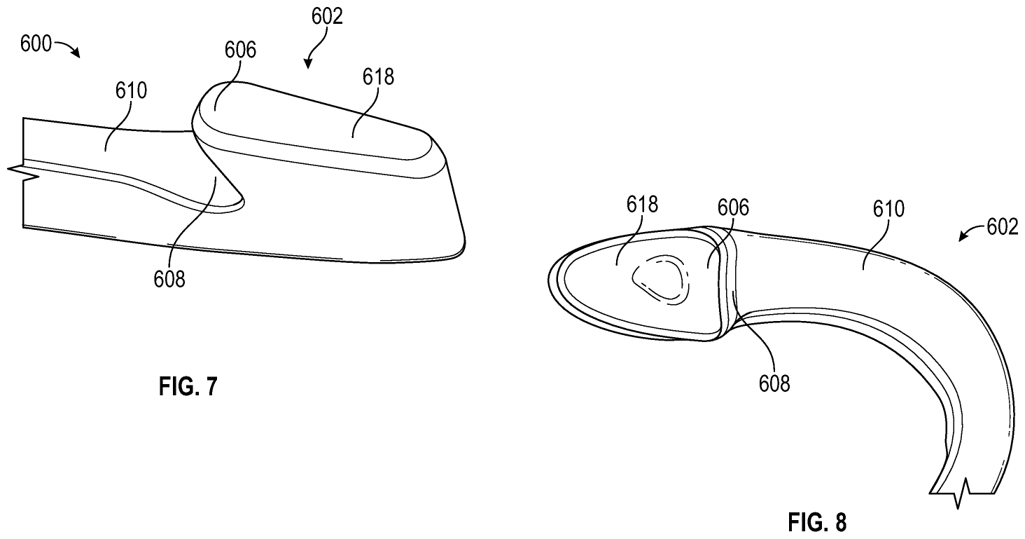

FIG. 7 illustrates a bottom perspective view of a portion the oral device 600. FIG. 8 illustrates a bottom view of a portion the oral device 600. FIGS. 7 and 8 are discussed together below. The oral device 600 of FIGS. 7 and 8 can be consistent with the oral device 600 discussed above. FIGS. 7 and 8 show additional details of the oral device 600. For example, FIGS. 7 and 8 show that the projection 606 can extend from the body 610 and can form a pad 618 that can be located at a posterior portion of the superior guard 602.

The pad 618 can be relatively wider and flatter than other portions of the body 610 such that the pad 618 can form a surface for engagement with molars of the dentition, which can help reduce tooth contact during grinding and can help reduce damage to the TMJ caused by clenching. Optionally, the pad 618 can be formed of a different material than the body 612. For example, the body 610 can be made of polyethylene-polyvinylacetate copolymer (EVA) and the pad 618 can be made of EVA that has a lower Shore value or is softer. In other examples, the body 610 can be made of EVA (or other thermo-polymer) and the pad 618 can be made of one or more of EVA, polyethylene terephthalate glycol (PETG), polyurethanes (TPU), polyolefin, polyurethanes, acrylic resins, or the like. The pad 618 can also optionally include air inclusions to help absorb shock. The pad 618 (optionally including air inclusions) can also be relatively larger and can therefore help to advance the mandible and the inferior dentition.

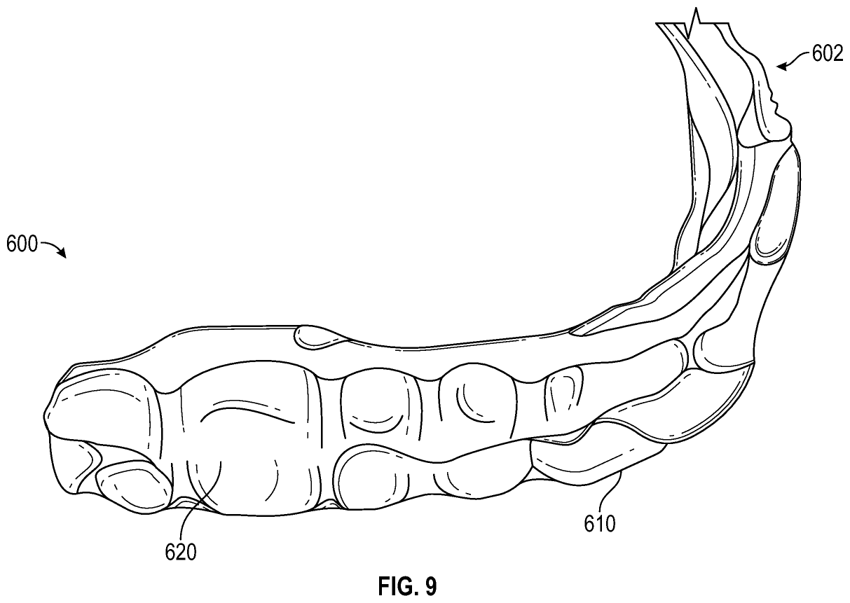

FIG. 9 illustrates a top view of a portion the oral device 600. The oral device 600 of FIG. 9 can be consistent with the oral device 600 discussed above. FIG. 9 shows additional details of the oral device 600. For example, FIG. 9 shows that tooth depressions can be formed into the body 610 of the superior guard 602 (and optionally of the inferior guard 604).

### NOTES AND EXAMPLES

The following, non-limiting examples, detail certain aspects of the present subject matter to solve the challenges and provide the benefits discussed herein, among others.

Example 1 is an oral device for use by a patient during sleep, the device comprising: a first guard configured for insertion into an oral cavity of the patient, the first guard comprising: a first body configured to engage a first set of a dentition of the patient; and a first recess extending into the first body, the first recess configured to align with a first set of occlusal surfaces of the first set of the dentition upon insertion of the first guard; and a second guard configured for insertion into the oral cavity of the patient, the second guard comprising: a second body configured to engage a second set of the dentition; and a first projection extending from the second body, the first projection to align with a first set of occlusal surfaces of the second set of the dentition upon insertion of the second guard, and the first projection insertable into the first recess to position the second guard with respect to the first guard and to position the first set of the dentition with respect to the second set of the dentition.

In Example 2, the subject matter of Example 1 optionally includes wherein the first set of the dentition is a superior set of the dentition and wherein the second set of the dentition is an inferior set of the dentition.

In Example 3, the subject matter of any one or more of Examples 1-2 optionally include wherein the first set of the dentition is an inferior set of the dentition and wherein the second set of the dentition is a superior set of the dentition.

In Example 4, the subject matter of any one or more of Examples 1-3 optionally include wherein the first guard includes a second recess configured to align with a second set of occlusal surfaces of the first set of the dentition upon insertion of the first guard, and wherein the second guard includes a second projection to align with a second set of occlusal surfaces of the second set of the dentition upon insertion of the second guard, and the second projection insertable into the second recess to position the second guard with respect to the first guard and to, together with the first projection and the first recess, position the first set of the dentition with respect to the second set of the dentition.

In Example 5, the subject matter of any one or more of Examples 1˜4 optionally include wherein the first projection is angled posteriorly with respect to the oral cavity.

In Example 6, the subject matter of any one or more of Examples 1-5 optionally include wherein the first projection is angled anteriorly with respect to the oral cavity.

In Example 7, the subject matter of any one or more of Examples 1-6 optionally include wherein the first projection and the first recess are configured to maintain a gap between superior incisors and inferior incisors of the dentition.

Example 8 is an oral device insertable into an oral cavity to engage a dentition of a patient, the device comprising: a first guard insertable into the oral cavity, the first guard comprising: a first lateral extension engageable with a first set of occlusal surfaces of a first set of the dentition and a second lateral extension engageable with a second set of occlusal surfaces of the first set of the dentition; a first recess extending into the first lateral extension and a second recess extending into the second lateral extension, the first recess and the second recess alignable with the first set of occlusal surfaces and the second set of occlusal surfaces of the first set of the dentition, respectively, upon insertion of the first guard; and a second guard insertable into the oral cavity, the second guard comprising: a third lateral extension engageable with a first set of occlusal surfaces of a second set of the dentition and a fourth lateral extension engageable with a second set of occlusal surfaces of the second set of the dentition; a first projection extending from the third lateral extension and a second projection extending from the fourth lateral extension, the first projection and the second projection alignable with the first set of occlusal surfaces and the second set of occlusal surfaces of the second set of the dentition, respectively, upon insertion of the second guard, and the first projection and the second projection insertable into the first recess and the second recess, respectively, to position the second guard with respect to the first guard and to position the first set of the dentition with respect to the second set of the dentition.

In Example 9, the subject matter of Example 8 optionally includes wherein the first set of the dentition is a superior set of the dentition and wherein the second set of the dentition is an inferior set of the dentition.

In Example 10, the subject matter of any one or more of Examples 8-9 optionally include wherein the first set of the dentition is an inferior set of the dentition and wherein the second set of the dentition is a superior set of the dentition.

Example 11 is an oral device insertable into an oral cavity to engage a dentition of a patient, the device comprising: a first guard insertable into the oral cavity, the first guard comprising: a first lateral extension engageable with a first set of occlusal surfaces of a first set of the dentition and a second lateral extension engageable with a second set of occlusal surfaces of the first set of the dentition; a first plurality of recesses extending into the first lateral extension and a second plurality of recesses extending into the second lateral extension; and a second guard insertable into the oral cavity, the second guard comprising: a third lateral extension engageable with a first set of occlusal surfaces of a second set of the dentition and a fourth lateral extension engageable with a second set of occlusal surfaces of the second set of the dentition; a first projection extending from the third lateral extension and a second projection extending from the fourth lateral extension, the first projection and the second projection insertable into one recess of the first plurality of recesses and one recess of the second plurality of recesses, respectively, to position the second guard with respect to the first guard and to position the first set of the dentition with respect to the second set of the dentition.

In Example 12, the subject matter of Example 11 optionally includes wherein the first plurality of recesses and the second plurality of recesses are alignable with the first set of occlusal surfaces and the second set of occlusal surfaces of the first set of the dentition, respectively, upon insertion of the first guard.

In Example 13, the subject matter of Example 12 optionally includes wherein the first projection and the second projection are alignable with the first set of occlusal surfaces and the second set of occlusal surfaces of the second set of the dentition, respectively, upon insertion of the second guard.

Example 14 is an oral device for use by a patient during sleep, the device comprising: a first guard configured for insertion into an oral cavity of the patient, the first guard comprising: a first body configured to engage a first set of a dentition of the patient; and a first projection extending from the first body, the first projection configured to align with a first set of occlusal surfaces of the first set of the dentition upon insertion of the first guard; and a second guard configured for insertion into the oral cavity of the patient, the second guard comprising: a second body configured to engage a second set of the dentition; and a second extending from the second body, the first second to align with a first set of occlusal surfaces of the second set of the dentition upon insertion of the second guard, and the first projection and the second projection engageable to position the second guard with respect to the first guard and to position the first set of the dentition with respect to the second set of the dentition.

In Example 15, the subject matter of Example 14 optionally includes wherein the first projection and the second projection are configured to interlock to cause or promote mandibular movement or displacement when the first guard and the second guard are inserted to engage the first set of the dentition and the second set of the dentition, respectively.

In Example 16, the subject matter of Example 15 optionally includes wherein the first guard includes a third projection configured to align with a second set of occlusal surfaces of the first set of the dentition upon insertion of the first guard, and wherein the second guard includes a fourth projection to align with a second set of occlusal surfaces of the second set of the dentition upon insertion of the second guard, and the second projection engageable with the fourth projection to position the second guard with respect to the first guard and to, together with the first projection and the second projection, position the first set of the dentition with respect to the second set of the dentition.

In Example 17, the subject matter of Example 16 optionally includes wherein the third projection is engageable with the fourth projection to, together with the first projection and the second projection, cause or promote anterior mandibular movement or displacement when the first guard and the second guard are inserted to engage the first set of the dentition and the second set of the dentition, respectively.

In Example 18, the subject matter of any one or more of Examples 14-17 optionally include wherein the first projection and the second projection are configured to form and maintain a gap between superior incisors and inferior incisors of the dentition when the first guard and the second guard are inserted to engage the first set of the dentition and the second set of the dentition, respectively.

In Example 19, the subject matter of any one or more of Examples 14-18 optionally include wherein the second set of the dentition an inferior set of the dentition, wherein the second guard is configured to engage the inferior dentition, and wherein the first projection is angled anteriorly to posteriorly with respect to the oral cavity.

In Example 20, the subject matter of Example 19 optionally includes wherein the first projection is angled anteriorly to posteriorly with respect to the oral cavity at an angle between 30 and 45 degrees.

In Example 22, an oral device comprises a first component, comprising a first portion, a second portion, and a third portion, the second portion extending from a first side of the first portion and the second portion extending from a second side of the first portion opposite the first side, the first through third portions configured to matingly and removably engage a first set of a patient's dentition and a second component, comprising a first portion, a second portion, and a third portion, the second portion extending from a first side of the first portion and the second portion extending from a second side of the first portion opposite the first side, the first through third portions configured to matingly and removably engage a second set of a patient's dentition. In this oral device, the first component comprises a first feature and the second component comprises a second feature in a position corresponding to a position of the first feature of the first portion of the first component when the first component and the second component are positioned in opposition to respectively engage the first set of the patient's dentition and the second set of the patient's dentition. In this oral device, the first feature and the second feature are dimensioned to matingly engage one another, wherein the first portion of each of the first component and the second component correspond to occlusal and/or incisal surfaces the first set of the patient's dentition and the second set of the patient's dentition, wherein the second portion of each of the first component and the second component correspond to facial (buccal or labial) surfaces the first set of the patient's dentition and the second set of the patient's dentition, and wherein the third portion of each of the first component and the second component correspond to lingual surfaces the first set of the patient's dentition and the second set of the patient's dentition. Engagement of the first feature and the second feature causes a restriction of translation of the first component and the second component in at least one direction, establishes a predetermined angle between the first component and the second component, or both causes a restriction of translation of the first component and the second component in at least one direction and establishes a predetermined angle between the first component and the second component. In exemplary aspects of this oral device, the first feature formed in the first portion of the first component and the second feature formed in the first portion of the second component are formed over the corresponding occlusal surfaces of the first set of the patient's dentition and the second set of the patient's dentition or, stated differently, are formed inwardly of the second portion and third portion of the first component and the second component (inwardly of facial (buccal or labial) and lingual surfaces, as applicable, of the first set of the patient's dentition and the second set of the patient's dentition).

In Example 23, the first feature of the first component of Example 22 comprises a recess and the second feature of the second component comprises a protrusion extending outwardly from the first portion of the second component.

In Example 24, the first feature of the first component of Example 22 comprises a plurality of recesses and the second component comprises a plurality of protrusions extending outwardly from the first portion of the second component in positions corresponding to the positions of the recesses of the first portion of the first component when the first component and the second component are positioned in opposition.

In Example 25, the first feature of the first component of Example 22 comprises a first recess and a first protrusion, which may be part of a unitary first feature or which may be spaced apart along the first portion of the first component and the second feature of the second component comprises a first recess and a first protrusion, which may be part of a unitary second feature or which may be spaced apart along the first portion of the second component wherein the first recess of the first component is configured to matingly engage the second protrusion of the second component and the second recess of the second component is configured to matingly engage the first protrusion of the first component when the first component and the second component are positioned in opposition.

In Example 26, the first component and the second component of any of Examples 22-25 are curvilinear.

In Example 27, the first component and the second component of any of Examples 22-26 are discontinuous.

In Example 28, the apparatuses or method of any one or any combination of Examples 1-27 can optionally be configured such that all elements or options recited are available to use or select from.

The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. § 1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

1. An oral device for use by a patient during sleep, the device comprising:
a first guard configured for insertion into an oral cavity of the patient, the first guard comprising:
a first body configured to engage a first set of a dentition of the patient; and
a first recess extending into the first body, the first recess configured to align with a first set of occlusal surfaces of the first set of the dentition upon insertion of the first guard; and
a second guard configured for insertion into the oral cavity of the patient, the second guard comprising:
a second body configured to engage a second set of the dentition; and
a first projection extending from the second body, the first projection to align with a first set of occlusal surfaces of the second set of the dentition upon insertion of the second guard, and the first projection insertable into the first recess to position the second guard with respect to the first guard and to position the first set of the dentition with respect to the second set of the dentition.

2. The oral device of claim 1, wherein the first set of the dentition is a superior set of the dentition and wherein the second set of the dentition is an inferior set of the dentition.

3. The oral device of claim 1, wherein the first set of the dentition is an inferior set of the dentition and wherein the second set of the dentition is a superior set of the dentition.

4. The oral device of claim 1, wherein the first guard includes a second recess configured to align with a second set of occlusal surfaces of the first set of the dentition upon insertion of the first guard, and wherein the second guard includes a second projection to align with a second set of occlusal surfaces of the second set of the dentition upon insertion of the second guard, and the second projection insertable into the second recess to position the second guard with respect to the first guard and to, together with the first projection and the first recess, position the first set of the dentition with respect to the second set of the dentition.

5. The oral device of claim 1, wherein the first projection is angled posteriorly with respect to the oral cavity.

6. The oral device of claim 1, wherein the first projection is angled anteriorly with respect to the oral cavity.

7. The oral device of claim 1, wherein the first projection and the first recess are configured to maintain a gap between superior incisors and inferior incisors of the dentition.

8. An oral device insertable into an oral cavity to engage a dentition of a patient, the device comprising:
a first guard insertable into the oral cavity, the first guard comprising:
a first lateral extension engageable with a first set of occlusal surfaces of a first set of the dentition and a second lateral extension engageable with a second set of occlusal surfaces of the first set of the dentition;
a first recess extending into the first lateral extension and a second recess extending into the second lateral extension, the first recess and the second recess alignable with the first set of occlusal surfaces and the second set of occlusal surfaces of the first set of the dentition, respectively, upon insertion of the first guard; and
a second guard insertable into the oral cavity, the second guard comprising:
a third lateral extension engageable with a first set of occlusal surfaces of a second set of the dentition and a fourth lateral extension engageable with a second set of occlusal surfaces of the second set of the dentition;
a first projection extending from the third lateral extension and a second projection extending from the fourth lateral extension, the first projection and the second projection alignable with the first set of occlusal surfaces and the second set of occlusal surfaces of the second set of the dentition, respectively, upon insertion of the second guard, and the first projection and the second projection insertable into the first recess and the second recess, respectively, to position the second guard with respect to the first guard and to position the first set of the dentition with respect to the second set of the dentition.

9. The oral device of claim 8, wherein the first set of the dentition is a superior set of the dentition and wherein the second set of the dentition is an inferior set of the dentition.

10. The oral device of claim 8, wherein the first set of the dentition is an inferior set of the dentition and wherein the second set of the dentition is a superior set of the dentition.

11. An oral device insertable into an oral cavity to engage a dentition of a patient, the device comprising:
a first guard insertable into the oral cavity, the first guard comprising:
a first lateral extension engageable with a first set of occlusal surfaces of a first set of the dentition and a second lateral extension engageable with a second set of occlusal surfaces of the first set of the dentition;
a first plurality of recesses extending into the first lateral extension and a second plurality of recesses extending into the second lateral extension; and
a second guard insertable into the oral cavity, the second guard comprising:
a third lateral extension engageable with a first set of occlusal surfaces of a second set of the dentition and a fourth lateral extension engageable with a second set of occlusal surfaces of the second set of the dentition;
a first projection extending from the third lateral extension and a second projection extending from the fourth lateral extension, the first projection and the second projection insertable into one recess of the first plurality of recesses and one recess of the second plurality of recesses, respectively, to position the second guard with respect to the first guard and to position the first set of the dentition with respect to the second set of the dentition.

12. The oral device of claim 11, wherein the first plurality of recesses and the second plurality of recesses are alignable with the first set of occlusal surfaces and the second set of occlusal surfaces of the first set of the dentition, respectively, upon insertion of the first guard.

13. The oral device of claim 12, wherein the first projection and the second projection are alignable with the first set of occlusal surfaces and the second set of occlusal surfaces of the second set of the dentition, respectively, upon insertion of the second guard.

14. An oral device for use by a patient during sleep, the device comprising:
a first guard configured for insertion into an oral cavity of the patient, the first guard comprising:
a first body configured to engage a first set of a dentition of the patient; and
a first projection extending from the first body, the first projection configured to align with a first set of occlusal surfaces of the first set of the dentition upon insertion of the first guard; and
a second guard configured for insertion into the oral cavity of the patient, the second guard comprising:
a second body configured to engage a second set of the dentition; and
a second extending from the second body, the first second to align with a first set of occlusal surfaces of the second set of the dentition upon insertion of the second guard, and the first projection and the second projection engageable to position the second guard with respect to the first guard and to position the first set of the dentition with respect to the second set of the dentition.

15. The oral device of claim 14, wherein the first projection and the second projection are configured to interlock to cause or promote mandibular movement or displacement when the first guard and the second guard are inserted to engage the first set of the dentition and the second set of the dentition, respectively.

16. The oral device of claim 15, wherein the first guard includes a third projection configured to align with a second set of occlusal surfaces of the first set of the dentition upon insertion of the first guard, and wherein the second guard includes a fourth projection to align with a second set of occlusal surfaces of the second set of the dentition upon insertion of the second guard, and the second projection engageable with the fourth projection to position the second guard with respect to the first guard and to, together with the first projection and the second projection, position the first set of the dentition with respect to the second set of the dentition.

17. The oral device of claim 16, wherein the third projection is engageable with the fourth projection to, together with the first projection and the second projection, cause or promote anterior mandibular movement or displacement when the first guard and the second guard are inserted to engage the first set of the dentition and the second set of the dentition, respectively.

18. The oral device of claim 14, wherein the first projection and the second projection are configured to form and maintain a gap between superior incisors and inferior incisors of the dentition when the first guard and the second guard are inserted to engage the first set of the dentition and the second set of the dentition, respectively.

19. The oral device of claim 14, wherein the second set of the dentition an inferior set of the dentition, wherein the second guard is configured to engage the inferior dentition, and wherein the first projection is angled anteriorly to posteriorly with respect to the oral cavity.

20. The oral device of claim 19, wherein the first projection is angled anteriorly to posteriorly with respect to the oral cavity at an angle between 30 and 45 degrees.
